(19) United States
(12) Patent Application Publication
(10) Pub. No.: US 2004/0223912 A1
(43) Pub. Date: Nov. 11, 2004

(54) Compositions and methods for non-invasive imaging of soluble beta-amyloid

(76) Inventors: Michael Christopher Montalto, Colonie, NY (US); Eric Dustin Agdeppa, Latham, NY (US); Tiberiu Mircea Siclovan, Rexford, NY (US); Amy Casey Williams, Clifton Park, NY (US)

Correspondence Address: Raymond E. Farrell, Esq., Carter, DeLuca, Farrell & Schmidt, LLP, Suite 225, 445 Broad Hollow Road, Melville, NY, 11747, US

(21) Appl. No.: 10/431,202

(22) Filed: May 7, 2003

(51) Int. Cl. A61K 51/00; A61K 49/00

(52) U.S. Cl. 424/1.49; 424/9.6; 530/391.1

(57) ABSTRACT

A method for assessing levels of soluble A-beta as an indicator of Alzheimer's disease, and other amyloid-related diseases, in vivo which employs an imaging agent binds specifically to soluble A-beta and is labeled for detection.

### TECHNICAL FIELD

The present disclosure relates to the detection of soluble beta-amyloid and the measurement of its local concentration in the brain of a subject without invasive procedures.

### BACKGROUND OF THE INVENTION

The main histopathological characteristics of Alzheimer's disease (“AD”) is the presence of neuritic plaques and tangles combined with associated inflammation in the brain. It is known that plaques are composed mainly of deposited (or insoluble in aqueous solution) fibrillar forms of the beta-amyloid (“A-beta”) peptide. The formation of fully fibrillar aggregated A-beta peptide is a complex process that is initiated by the cleavage of the amyloid precursor protein (“APP”). After cleavage of APP, the monomeric form of A-beta can associate with other monomers, presumably through hydrophobic interactions and/or domain swapping, to form dimers, trimers and higher order oligomers. Oligomers of A-beta can further associate to form protofibrils and eventual fibrils, which is the main constituent of neuritic plaques. It has recently been shown that soluble oligomers (soluble in aqueous buffer) of A-beta may contribute significantly to neuronal dysfunction. In fact, animal models suggest that simply lowering the amount of soluble A-beta peptide, without affecting the levels of A-beta in plaques, may be sufficient to improve cognitive function.

Presently, the only definitive method of AD diagnosis is postmortem examination of brain for the presence of plaques and tangles. The antemortem diagnosis of AD is difficult, especially during the early stages, as AD symptoms are shared among a spectrum of other dementias. Currently, AD diagnosis is achieved using simple cognitive tests designed to test a patient's mental capacity such as, for example, the ADAS-cog (Alzheimer's disease assessment scale—cognitive subscale) or MMSE (Mini-mental state examination). The subjective nature and inherent patient variability is a major shortcoming of diagnosing AD by such means. The fact that AD cannot be accurately diagnosed early creates a formidable challenge for pharmaceutical companies that aim to test anti-A-beta drugs as therapy to slow or halt AD pathogenesis. Furthermore, even if AD could be detected early and patients could be treated with A-beta lowering compounds, there is currently no way to know if the therapy is clinically efficacious. Therefore, a significant need exists to develop methods of measuring the soluble A-beta peptide levels locally in the brain.

Diagnosing AD by directly measuring levels of beta-amyloid noninvasively has been attempted by the targeted imaging of senile plaques. This approach fails as a specific measure of soluble A-beta peptide because current A-beta targeted imaging agents are directed at insoluble aggregates that are characteristic of A-beta fibrillar deposits in the brain. Further, targeted imaging of plaques may not provide early diagnosis, as large plaque burden is mostly associated with mid to late stage disease. Moreover, it has not been shown that current anti-A-beta therapies will affect fibrillar deposits appreciably to detect by imaging techniques at clinically relevant time points.

Alternatively, in vitro measures of A-beta may be specific for soluble A-beta in the cerebral spinal fluid, but lacks the necessary selectivity for local A-beta in the brain that is necessary for direct, accurate assessment of brain levels of soluble A-beta species. To date, the targeted non-invasive measurement and imaging of soluble A-beta peptide species (including monomer, dimers, trimers and n-oligomers) that exist in the central nervous system (“CNS”) have not been addressed.

### SUMMARY

This disclosure relates to a method of assessing in vivo the presence and quantity of A-beta by administering to a subject an imaging agent that binds to or otherwise reports on the presence or quantity of soluble A-beta and is labeled for detection. The compound is then non-invasively detected and measured by imaging modalities when incorporated as complex of the imaging agent bound to soluble A-beta. The compositions of the labeled imaging compounds that bind to or reports on soluble A-beta are also described.

In another aspect, methods of non-invasively diagnosing and assessing amyloid-related disease are described which include the steps of administering to a subject a labeled compound that has specific binding to soluble peptides related to amyloid and steps of determining the extent of specific binding.

In yet another aspect, methods of non-invasively assessing the therapeutic efficacy of therapies in a subject are described which include the steps of tracking the therapeutic modification of the proteolytic processing of amyloid precursor proteins and subsequently tailoring the administered dose of therapeutic agents in response to monitoring.

### DETAILED DESCRIPTION

The present disclosure relates to a method of non-invasively assessing levels of soluble A-beta to diagnose amyloid-related diseases, including Alzheimer's disease. This method qualitatively and quantitatively determines soluble A-beta levels in vivo. This method can also be used to determine the efficacy of related therapies used for amyloid-related diseases. To assess the soluble A-beta levels, a labeled diagnostic imaging agent is delivered to a subject. Typically, the subject is a mammal and can be human. The labeled imaging agent contains at least a chemical entity that binds to soluble A-beta and a chemical entity that emits a signal detectable by an imaging modality. The labeled imaging agent is delivered to a subject by a medically appropriate means. After allowing a clearance time according to the label chosen, the amount of imaging agent bound to soluble A-beta is determined by noninvasively measuring the emitted signal using an imaging modality. The visual and quantitative analyses of the resulting images provide an accurate assessment of the levels of soluble A-beta in the brain.

The chemical entity of the imaging agent that binds to soluble A-beta can bind to monomers, dimers, trimers and/or oligomers comprised of a larger number of A-beta peptides up to 24 A-beta peptides. More specifically, the soluble A-beta species to which the imaging agent can bind include monomers, dimers, trimers, and oligomers of A-beta 1-38, A-beta 1-39, A-beta 1-40, A-beta 1-41, A-beta 1-42, A-beta 1-43 or any combination thereof. The A-beta peptide in soluble monomer or oligomer forms can be derived ex vivo, by recombinant means, or synthetically. The soluble A-beta includes monomeric and low oligomeric A-beta that is soluble in an aqueous solution. In some embodiments, the soluble A-beta is of a type that remains in the supernatant of aqueous solution after centrifugation at 15000 times gravity. In some embodiments, the soluble A-beta includes A-beta monomers and its aggregates that do not exhibit green birefringence when stained by Congo red.

The imaging agent that binds to soluble A-beta or otherwise reports on the presence of soluble A-beta can be derived from a natural source or be man made and be a small molecule, peptide, protein, enzyme, dendrimer, polymer, antibody or antibody fragment.

The term “small molecule” means a molecule having a molecular weight of equal to or less than about 5000 daltons. In certain embodiments the small molecule has a molecular weight in the range of 300 to 2000 daltons. As well known in the art, such compounds may be found in compound libraries, combinatorial libraries, natural products libraries, and other similar sources, and may further be obtained by chemical modification of compounds found in those libraries, such as by a process of medicinal chemistry as understood by those skilled in the art, which can be used to produce compounds having desired pharmacological properties.

Unlike the presently described imaging agents that bind to soluble A-beta, there are imaging agents and dyes that bind exclusively to insoluble deposits of A-beta or senile plaques. Small molecules that specifically bind to insoluble A-beta deposits include, for example, small molecular weight molecules, such as Congo red, Chrysamine G, methoxy-X04, TZDM, [¹¹C]6, IMSB, Thioflavin(e) S and T, TZDM, 1-BTA, benzathiozole derivatives, [¹²⁵ I]3, BSB, IMSB, styrylbenzene-derivatives, IBOX, benzoxazole derivatives, IMPY, pyridine derivatives, DDNP, FDDNP, FENE, dialkylaminonaphthyl derivatives, benzofuran derivatives, and derivatives thereof (see, e.g., U.S. Pat. Nos. 6,133,259; 6,168,776; 6,114,175.

Nucleic acid sequences and derivatives thereof have been shown to bind to insoluble senile plaques of A-beta, including mRNA for furin and amyloid precursor protein (“APP”).

Peptides also have been developed as imaging agents for insoluble deposits of A-beta and senile plaques. The sequence specific peptides that have been labeled for the purpose of imaging insoluble A-beta includes the labeled A-beta peptide itself, putrescine-gadolinium-A-beta peptide, radiolabeled A-beta, [¹¹¹In]A-beta, [¹²⁵I]A-beta, A-beta labeled with gamma emitting radioisotopes, A-beta-DTPA derivatives, radiolabeled putrescine, KVLFF-based ligands and derivatives thereof (see, e.g., International Pub. No. WO93/04194 and U.S. Pat. No. 6,331,440).

Inhibitors of aggregated A-beta have been suggested to disrupt the formation of these aggregates by interacting with soluble and/or insoluble fibrils of A-beta. Examples of inhibitors or anti-aggregation agents include peptides of A-beta, KVLFF-based ligands, small molecular weight compounds, carbon nanostructures, rifamycin, IDOX, acridone, benzofuran, apomorphine, and derivatives thereof.

Agents have also been know to promote aggregation—agents such as A-beta42, proteins, metals, small molecular weight compounds, and lipids. Agents that either promote aggregation or disaggregation of A-beta fibrils presumably interact with either soluble or insoluble A-beta or both, suggesting that developing compounds that exclusively bind A-beta is feasible.

Antibodies for A-beta are similar to KLVFF-derivative as they also interact with soluble and insoluble A-beta. Antibodies specific for soluble and insoluble A-beta can be prepared against a suitable antigen or hapten comprising the desired target epitope, such as the junction region consisting of amino acid residues 13-26 and/or the carboxy terminus consisting of amino acid residues 33-42 of A-beta. One suitable antibody to soluble A-beta is disclosed in Kayed, et al., Science, vol. 300, page 486, Apr. 18, 2003. Synthetic peptides can also be prepared by conventional solid phase techniques, coupled to a suitable immunogen, and used to prepare antisera or monoclonal antibodies by conventional techniques. Suitable peptide haptens typically will comprise at least five contiguous residues within A-beta and can include more than six residues. Synthetic polypeptide haptens can be produced by the Merrifield solid-phase synthesis technique in which amino acids are sequentially added to a growing chain (Merrifield (1963) J. Am. Chem. Soc. 85:2149-2156). Suitable antibodies include, for example, those of U.S. Pat. Nos. 5,811,310; 5,750,349; and 5,231,000, R1282, 21F12, 3D6, FCA3542, and monoclonal and polyclonal antibodies for A-beta 1-40, 1-42 and other isoforms. Certain imaging agents have been developed that can report on the specific presence of a target molecule without binding to that molecule. In such instances the imaging agents are considered “activatable” because their signal is activated or unactivated based on the presence of a specific target molecule. Examples of such agents have been used for MRI and optical imaging (Li W H, Parigi G, Fragai M, Luchinat C, Meade T J, Inorg Chem 2002 July 29;41(15):4018-24)(Louie A Y, Huber M M, Ahrens E T, Rothbacher U, Moats R, Jacobs R E, Fraser S E, Meade T J. Nat Biotechnol 2000 March ;18(3):321-5)(Weissleder R, Tung C H, Mahmood U, Bogdanov A Jr Nat Biotechnol 1999 April;17(4):375-8).

The chemical entity of the imaging agent that emits a detectable signal (also called a label) can be a radiolabel, a paramagnetic label, an optical label and the like. The type of imaging modality available will be an important factor in the selection of the label used for an individual subject. For example, a radiolabel must have a type of decay that is detectable by the available imaging modality. Suitable radioisotopes are well known to those skilled in the art and include beta-emitters, gamma-emitters, positron-emitters, and x-ray emitters. Suitable radioisotopes include ³H, ¹¹C, ¹⁴C, ¹⁸F, ³²P, ³⁵S, ¹²³I, ¹²⁵I, ¹³¹I, ⁵¹Cr, ³⁶CI, ⁵⁷Co, ⁵⁹Fe, ⁷⁵Se and ¹⁵²Eu. Isotopes of halogens (such as chlorine, fluorine, bromine and iodine), and metals including technetium, yttrium, rhenium and indium are also useful labels. Typical examples of metallic ions which can be bound are ^{99m}Tc, ¹²³I, ¹¹¹In, ¹³¹I, ⁹⁷Ru, ⁶⁷C, ⁶⁷Ga, ¹²⁵I, ⁶⁸Ga, ⁷²As, ⁸⁹Zr, and ²⁰¹Tl. For use with the present disclosure, radiolabels can be prepared using standard radiolabeling procedures well known to those skilled in the art. The disclosed compound can be radiolabeled either directly by incorporating the radiolabel directly into the compounds or indirectly by incorporating the radiolabel into the compounds through a chelating agent, where the chelating agent has been incorporated into the compounds. Such radiolabeling should also be reasonably stable, both chemically and metabolically, applying recognized standards in the art. Also, although the label can be incorporated in a variety of fashions with a variety of different radioisotopes, such radiolabeling should be carried out in a manner such that the high binding affinity and specificity of the unlabeled binding moiety is not significantly affected. Preferred radioisotopes for in vivo diagnostic imaging by positron emission tomography (“PET”) are 11C, 18F, 123I, and 125I. Typically, the labeled atom is introduced to the labeled compounds at a late stage of the synthesis. This allows for maximum radiochemical yields, and reduces the handling time of radioactive materials. When dealing with short half-life isotopes, an important consideration is the time required to conduct synthetic procedures, and purification methods. Protocols for the synthesis of radiolabeled compounds are described in Tubis and Wolf, Eds., “Radiopharmacy”, Wiley-Interscience, New York (1976); Wolf, Christman, Fowler, Lambrecht, “Synthesis of Radiopharmaceuticals and Labeled Compounds Using Short-Lived Isotopes”, in Radiopharmaceuticals and Labeled Compounds, Vol. 1, p. 345-381 (1973).

Paramagnetic labels can be metal ions are present in the form of metal complexes or metal oxide particles. Suitable paramagnetic isotopes include 157Gd, 55Mn, 162 Dy, 52Cr, and 56Fe. The paramagnetic label can be attached to the binding moiety by several approaches. One approach is direct attachment of one or more metal chelators to the binding moiety of the imaging agent. Alternatively, the binding portion of the imaging agent can be attached to a paramagnetic metal ion or heavy atom containing solid particle, or to an echogenic gas microbubble. A number of methods can be used to attach imaging agent, which specifically binds to soluble A-beta, to paramagnetic metal ion or heavy atom containing solid particles by one of skill in the art of the surface modification of solid particles. In general, the imaging agent is attached to a coupling group that react with a constituent of the surface of the solid particle. The coupling groups can be any of a number of silanes, and also include polyphosphonates, polycarboxylates, polyphosphates or mixtures thereof, which react with surface hydroxyl groups on the solid particle surface, as described, for example, in U.S. patent application publication 2002/0159947 and which can couple with the surface of the solid particles, as described in U.S. Pat. No. 5,520,904.

The imaging agent itself can be fluorescent or can be tagged with optical labels that are fluorophores, such as fluorescein, rhodamine, Texas Red, and derivatives thereof and the like. The labels can be chemiluminescent, such as green fluorescent protein, luciferin, dioxetane, and the like. These fluorophore probes are commercially-available, e.g., from Molecular Probes, Inc., Eugene, Oreg. The imaging agent that binds to soluble A-beta can be linked to the portion of the compound that emits a detectable signal by techniques known to those skilled in the art.

The labeled imaging agent can typically be administered to a patient in a composition comprising a pharmaceutical carrier. A pharmaceutical carrier can be any compatible, non-toxic substance suitable for delivery of the labeled A-beta binding compound to the patient, including sterile water, alcohol, fats, waxes, proteins, and inert solids may be included in the carrier. Pharmaceutically acceptable adjuvants (buffering agents, dispersing agent) can also be incorporated into the pharmaceutical composition. Carriers can contain a solution of the imaging agent or a cocktail thereof dissolved in an acceptable carrier, preferably an aqueous carrier. A variety of aqueous sterile carriers can be used, e.g., water, buffered water, 0.4% saline, 0.3% glycine and the like. The solutions must also be pyrogen-free, sterile, and generally free of particulate matter. The compositions can contain additional pharmaceutically acceptable substances as necessary to approximate physiological conditions such as pH adjusting and buffering agents, toxicity adjusting agents and the like, for example sodium acetate, sodium chloride, potassium chloride, calcium chloride, sodium lactate. The concentration of imaging agent in the composition solutions may vary as required. Typically, the concentration will be in trace amounts to as much as 5% by weight depending on the imaging modality and are selected primarily based on fluid volumes, and viscosities in accordance with the particular mode of administration selected. A typical composition for intravenous infusion can be made to contain 250 ml of sterile Ringer's solution and up to 100 mg of the imaging agent. The composition containing the imaging agent can be combined with a pharmaceutical composition and can be administered subcutaneously, intramuscularly or intravenously to patients suffering from, or at risk of, amyloid-related conditions.

The imaging agent is administered to a subject to determine the presence and amount of soluble amyloid in the subject. After administration, clearance time can, if desired, be permitted which allows the imaging agent to travel throughout the subject's body and bind to any available soluble A-beta whereas the unbound imaging agent passes through the subject's body. In a case where the imaging agent does not directly bind, but rather reports on the presence of the A-beta, sufficient time is allowed for a specific interaction to occur in which the reporter molecule is “activated”. The clearance time will vary depending on the label chosen for use and can range from 1 minute to 24 hours. The imaging agent is then detected noninvasively in the subject's body by an imaging modality. The imaging modality can include positron emission tomography (“PET”), optical, single photon emission computed tomography (“SPECT”), ultrasound, computed tomography (“CT”), and the like, depending on the label used, the modality available to medical personnel and the medical needs of the subject. Equipment and methods for the foregoing imaging modulations are those to those skilled in the art.

The imaging agent can be delivered and the imaging taken to determine the amount of soluble A-beta present in the subject's body as an indication of disease or pre-disease states. The levels of soluble A-beta can be indicative of pre-disease conditions and therapies toward removal of the soluble A-beta and/or its precursors can prevent or forestall the onset of an amyloid-related disease, such as Alzheimer's disease. The removal of soluble A-beta can also improve the condition of a subject that already exhibits clinical signs of disease.

In another aspect, the present methods can be used to determine the efficacy of therapies used in a subject. By using multiple images over time, the levels of A-beta can be tracked for changes in amount and location. This method can aid physicians in determining the amount and frequency of therapy needed by an individual subject. In this embodiment, an imaging agent in accordance with the present disclosure is administered and a baseline image is obtained. The therapy to be evaluated is administered to the subject either before or after a baseline images are obtained. After a pre-determined period of time, a second administration of an imaging agent in accordance with their disclosure is given. A second or more images are obtained. By qualitatively and quantitatively comparing the baseline and the second image, the effectiveness of the therapy being evaluated can be determined based on a decrease or increase of the signal intensity of the second image or additional images.

Although preferred and other embodiments of the invention have been described herein, further embodiments may be perceived by those skilled in the art without departing from the scope of the invention as defined by the following claims.

What is claimed is:

1. A method comprising:
administering to a subject an imaging agent that binds to a soluble A-beta and is labeled for detection; and
non-invasively detecting the imaging agent that is present as a complex of the imaging agent bound to soluble A-beta.

2. A method as in claim 1, wherein the soluble A-beta is selected from the group consisting of monomers, dimers, trimers, oligomers of up to 24 A-beta peptides, and combinations thereof.

3. A method as in claim 1, wherein the soluble A-beta to which the imaging agent binds is selected from the group consisting of monomers, dimers, trimers, and oligomers of A-beta 1-38, A-beta 1-39, A-beta 1-40, A-beta 1-41, A-beta 1-42, A-beta 1-43 and combinations thereof.

4. A method as in claim 1, wherein the soluble A-beta is selected from the group consisting of A-beta that does not exhibit green birefringence when stained by Congo red.

5. A method as in claim 1, wherein the imaging agent that binds to soluble A-beta is selected from the group consisting of small molecules, antibody fragments, nucleic acid, peptides, antibodies, dendrimers, proteins and polymers.

6. A method as in claim 1, wherein the imaging agent is labeled with a member selected from the group consisting of radioisotopes, paramagnetic particles and optical particles.

7. A method as in claim 1, wherein the imaging agent is labeled with a radioisotope selected from the group consisting of 3H, 11C, 14C, 18F, 32P, 35S, 123I, 125I, 131I 51Cr, 36CI, 57Co, 59Fe, 75Se and 152Eu.

8. A method as in claim 1, wherein the imaging agent is labeled with a paramagnetic particle selected from the group consisting of 157Gd, 55Mn, 162 Dy, 52Cr, and 56Fe.

9. A method as in claim 1, wherein the imaging agent comprises an optical label selected from the group consisting of fluorophores and chemiluminescent entities.

10. A method as in claim 1, wherein the step of non-invasive detection comprises generating and analyzing an image using a technique selected from the group consisting of positron emission tomography, magnetic resonance imaging, optical imaging, single photon emission computed tomography, ultrasound and x-ray computed tomography.

11. A method as in claim 1, wherein the step of non-invasive detection further comprises measuring the amount of imaging agent that is present as a complex of the imaging agent bound to soluble A-beta.

12. A method of assessing an amyloid -related disease comprising: administering to a subject having or suspected of having an amyloid-related disease, an imaging agent that specifically binds to a soluble beta-amyloid and is labeled to emit a detectable signal; and detecting the imaging agent bound to A-beta using non-invasive imaging.

13. A method as in claim 12, wherein the soluble A-beta is selected from the group consisting of monomers, dimers, trimers, oligomers of up to 24 A-beta peptides and combinations thereof.

14. A method as in claim 12, wherein the soluble A-beta is selected from the group of A-beta 1-38, A-beta 1-39, A-beta 1-40, A-beta 1-41, A-beta 1-42, A-beta 1-43 and combinations thereof.

15. A method as in claim 12, wherein the imaging agent that binds to soluble A-beta is selected from the group consisting of small-molecules, peptides, antibodies, dendrimers, proteins, polymers and antibody fragments.

16. A method as in claim 12, wherein the imaging agent comprises a label selected from the group consisting of radioisotopes, paramagnetic particles and optical particles.

17. A method as in claim 12, wherein the imaging agent comprises a label selected from the group consisting of 3H, 11C, 14C, 18F, 32P, 35S, 123I, 125I, 131I 51Cr, 36CI, 57Co, 59Fe, 75Se and 152Eu.

18. A method as in claim 12, wherein the imaging agent comprises a label selected from the group consisting of 157Gd, 55Mn, 162 Dy, 52Cr, and 56Fe.

19. A method as in claim 12, wherein the imaging agent comprises an optical label selected from the group consisting of fluorophores and chemiluminescent entities.

20. A method as in claim 12, wherein the amyloid-related disease is Alzheimer's disease.

21. A method as in claim 12, wherein the step of detecting comprises noninvasively measuring the level of the imaging agent within the subject.

22. A method as in claim 12 wherein the step of detecting comprises imaging the brain of the subject.

23. A method of evaluating the effectiveness of a therapy comprising:
administering to a subject a first dose of a composition comprising an imaging agent that binds to soluble A-beta and is labeled for detection and a pharmaceutical carrier;
non-invasively obtaining a baseline measurement of the imaging agent within the subject;
administering to the subject a therapy to be evaluated;
administering to the subject a second dose of said composition;
non-invasively obtaining a second measurement of the imaging agent within the subject; and
comparing the two or more measurements separated in time, wherein an increase or decrease in the amount of the imaging agent present indicates the efficacy of the therapy.

24. A method as in claim 23 wherein the therapy to be evaluated is administered before administration of the first dose of the composition.

25. A method as in claim 23 wherein the first dose of the composition comprises the imaging agent in an amount ranging from a trace amount to about 100 mg.

26. An imaging composition comprising:
an imaging agent that binds to soluble A-beta and is labeled for detection; and
a pharmaceutically acceptable carrier.

27. A method comprising:
administering to a subject an imaging agent that reports on soluble A-beta and carries a molecule or element that can be detected by imaging methods;
non-invasively detecting the imaging agent that becomes activated when soluble A-beta is present.

28. A method as in claim 27, wherein the soluble A-beta is selected from the group consisting of monomers, dimers, trimers, oligomers of up to 24 A-beta peptides, and combinations thereof.

29. A method as in claim 27, wherein the soluble A-beta to which activates the imaging agent is selected from the group consisting of monomers, dimers, trimers, and oligomers of A-beta 1-38, A-beta 1-39, A-beta 1-40, A-beta 1-41, A-beta 1-42, A-beta 1-43 and combinations thereof.

30. A method as in claim 27, wherein the soluble A-beta is selected from the group consisting of A-beta that does not exhibit green birefringence when stained by Congo red.

31. A method as in claim 27, wherein the imaging agent is selected from the group consisting of small molecules, antibody fragments, nucleic acid, peptides, antibodies, dendrimers, proteins and polymers.

32. A method as in claim 27, wherein the imaging agent is labeled with a member selected from the group consisting of radioisotopes, paramagnetic particles and optical particles.

33. A method as in claim 27, wherein the imaging agent is labeled with a radioisotope selected from the group consisting of 3H, 11C, 14C, 18F, 32P, 35S, 123I, 125I, 131I 51Cr, 36CI, 57Co, 59Fe, 75Se and 152Eu.

34. A method as in claim 27, wherein the imaging agent is labeled with a paramagnetic particle selected from the group consisting of 157Gd, 55Mn, 162 Dy, 52Cr, and 56Fe.

35. A method as in claim 27, wherein the imaging agent comprises an optical label selected from the group consisting of fluorophores and chemiluminescent entities.

36. A method as in claim 27, wherein the step of non-invasive detection comprises generating and analyzing an image using a technique selected from the group consisting of positron emission tomography, magnetic resonance imaging, optical imaging, single photon emission computed tomography, ultrasound and x-ray computed tomography.

37. A method as in claim 27, wherein the step of non-invasive detection further comprises measuring the amount of imaging agent that is activated by soluble A-beta.
